(19) United States
(12) Patent Application Publication
(10) Pub. No.: US 2024/0050231 A1
(43) Pub. Date: Feb. 15, 2024

(54) Acetabular spacer device comprising a pharmaceutical substance

(71) Applicant: Cossington Limited, Kingston upon Thames (GB)

(72) Inventors: Augusto Magagnoli, Cervia (Ravenna) (IT); Robert Michael Meneghini, McCordsville, IN (US); Bryan Donald Springer, Charlotte, NC (US); Scott Matthew Sporer, Wheaton, IL (US); Stephen Joseph Incavo, Houston, TX (US)

(21) Appl. No.: 18/495,030

(22) Filed: Oct. 26, 2023

Related U.S. Application Data
(63) Continuation of application No. 15/733,524, filed on Aug. 19, 2020, now Pat. No. 11,833,051.

(51) Int. Cl. A61F 2/30

(52) U.S. Cl. CPC A61F 2/30724; A61F 2002/30672; A61F 2002/30677; A61F 2002/30879; A61F 2002/3425

(57) ABSTRACT

An acetabular spacer device, of a type that is temporary and disposable, adapted to be implanted in use in a bone cavity placed at a joint of the human body, such as a hip or shoulder joint, has a cup-like shape, substantially hemispherical, and includes a first convex surface, adapted to be positioned at the bone cavity, a second concave surface, which defines a cavity, further includes at least one pharmaceutical or medical substance, such as at least one antibiotic, adapted to treat during use an ongoing infection in the bone cavity.

### TECHNICAL FIELD OF THE INVENTION

The present invention relates to an acetabular spacer device, adapted to be implanted in a corresponding cavity of the human body.

In particular, the acetabular spacer device is, of a temporary and disposable type, and is used for the treatment of a bone site subject to infection, in view of the implant of a prosthesis or of a new prosthesis.

### PRIOR ART

The term “acetabolum” relates in general to a recess or cavity of the human body in which the condyle of a nearby moving organ is inserted.

For example, the acetabulum present in the pelvis at the hip joint is also called “cup” and is formed by the fusion of ileum, ischium and pubis; it constitutes a joint cavity and is able to articulate with the head of the femur (coxofemoral joint).

The glenoid cavity of the scapula is also present in the human body: it is a shallow, articular surface that is articulated with the head of the humerus (glenohumeral joint). Following traumas or pathologies of the joints of the human body, it is possible to intervene by implanting a permanent prosthesis.

It can also happen that infections occur on the joint site and in this case, both if a prosthesis is already present on the site and if it is the patient's bone tissue, it is necessary to eradicate the bacterial outbreak by treatment with pharmaceutical or medical substances, such as for example antibiotics (possibly removing the present prosthesis).

The spacer device according to the present invention aims at this objective, with particular reference to the bone tissues surrounding the articular cavity, both in the case of infection occurring in the patient's bone tissue and following the implantation of a permanent prosthesis.

### OBJECTS OF THE INVENTION

The task of the present invention is to improve the prior art.

Within the scope of this technical task, it is an object of the present invention to provide an acetabular spacer device for the treatment of an infection occurring at a joint cavity of the human body.

A further object of the present invention is to provide an acetabular spacer device adapted to restore the shape of the cavity on which it is implanted and thus improve the joint with the head of a femur or of a humerus or with the head of respective prosthetic components or of hip or shoulder spacer devices.

Another object of the present invention is to provide an acetabular spacer device having shape or dimensions corresponding to the anatomical shape or dimensions of the patient.

According to one aspect of the present invention, an acetabular spacer device according to claim 1 is provided.

The dependent claims refer to preferred and advantageous embodiments of the invention.

### BRIEF DESCRIPTION OF THE DRAWINGS

Further features and advantages of the present invention will appear more clearly from the detailed description of a preferred but non-exclusive embodiment of an acetabular spacer device, given by way of a non-limiting example in the accompanying drawings, in which:

FIG. 1 is a perspective bottom view of the acetabular spacer device according to a first version of the invention;

FIG. 2 is a perspective top view of the acetabular spacer device in FIG. 1;

FIG. 3 is a perspective top view of the acetabular spacer device according to a second version of the invention;

FIG. 4 is a perspective top view of the acetabular spacer device according to a third version of the invention;

FIG. 5 is a top view of the acetabular spacer device in FIGS. 1 and 2;

FIG. 6 is a front view of the acetabular spacer device in FIGS. 1, 2 and 5;

FIG. 7 is a side view of the acetabular spacer device in FIGS. 1, 2 and 5;

FIG. 8 is a sectional view of the acetabular spacer device along the section plane VIII-VIII in FIG. 5;

FIG. 9 is a sectional view of the acetabular spacer device along the section plane IX-IX in FIG. 5;

FIG. 10 is a top view of the acetabular spacer device in FIG. 3;

FIG. 11 is a front view of the acetabular spacer device in FIGS. 3 and 10;

FIG. 12 is a sectional view of the acetabular spacer device along the section plane XII-XII in FIG. 10;

FIG. 13 is a top view of the acetabular spacer device in FIG. 4;

FIG. 14 is a front view of the acetabular spacer device in FIGS. 4 and 13;

FIG. 15 is a sectional view of the acetabular spacer device along the section plane XV-XV in FIG. 13.

### EMBODIMENTS OF THE INVENTION

With reference to the figures, 1 globally indicates an acetabular spacer device according to the present invention.

The acetabular spacer device 1 is a disposable and temporary spacer device, suitable for treating an infection occurred at the implantation site.

The acetabular spacer device 1 is adapted to be implanted in use in a bone cavity located at a joint of the human body, such as a hip or shoulder joint, thus for example at the cotyloid cavity of the joint of the hip or at the glenoid cavity of the shoulder joint. In the illustrated images, in particular, an acetabular spacer device 1 is shown for the treatment of a hip joint and/or for the replacement of a hip prosthesis, but the present invention can also be referred to devices for treating other types of bone sites and/or for the replacement of other types of prostheses, for example, seats or prostheses of the humerus, seats or prostheses of ankle, elbow, etc.

The acetabular spacer device 1 according to the invention is suitable for being implanted, if necessary, in place of a permanent prosthesis previously inserted at the respective joint or seat, which has undergone infection.

Furthermore, the acetabular spacer device 1 is adapted to articulate with the head of the femur, with the head of the humerus, with a corresponding prosthetic component or with a corresponding spacer device, for example with the stem component of a respective joint.

The acetabular spacer device 1 according to the present invention includes at least one pharmaceutical or medical substance, such as at least one antibiotic, capable of combating an infection in place at the implantation site.

The acetabular spacer device 1 has a cup shape, that is to say, a substantially hemispherical shape.

In particular, the acetabular spacer device 1 has a first surface 2, that is convex, suitable for being positioned at the acetabular cavity of the patient's bone, and a second surface 3, that is concave.

The second surface 3 is adapted to articulate with the head of a femur, of a humerus or with corresponding components of a permanent prosthesis or a spacer device, for example with the femoral or humeral stem of a spacer device or of a permanent prosthesis.

At the first surface 2, the acetabular spacer device 1 comprises a plurality of elongated projections or recesses 4, extending outwardly or inwardly (with respect to the first surface 2) of the acetabular spacer device 1 itself.

In particular, the first surface 2 has such a shape and dimensions as to adapt to the patient's acetabulum or bone cavity, since it has a shape substantially complementary to that of the bone cavity.

The second surface 3, on the other hand, has such a shape and dimensions that it allows the housing and the articulation of the head of the respective component, in consideration of the joint in which the acetabular spacer device 1 is in use inserted.

The second surface 3, moreover, is completely smooth, precisely to facilitate the articulation and the rotation/sliding of the head of the respective component.

Entering more in detail, the acetabular spacer device 1 has—as said—a cup-shaped configuration, in which the first and second surfaces 2, 3 are substantially semi-spherical.

In a version of the invention, the first surface 2 and the second surface 3 are coaxial, and in particular have the same central symmetry axis X, which corresponds to that of the acetabular spacer device 1.

The acetabular spacer device 1, according to a version of the invention, comprises a base 5, perpendicular to the central symmetry axis X of the device itself.

The base 5 is formed by a base ring 5a which connects the first surface 2 and the second surface 3, in particular the terminal circular perimeter of the first end 2 and the terminal circular perimeter of the second end 3.

The base 5 also delimits an opening 5b which delimits the gap or inlet opening towards the second surface 3 and/or the gap or inlet opening of the cavity 7 defined by the second surface 3 which is convex.

The cavity 7 has a substantially hemispherical conformation.

The opening 5b, as well as the base 5, have a substantially circular or annular shape.

Therefore, the first surface 2 and the second surface 3 have a shape corresponding to that of a spherical base cap 5.

The area of connection between the base ring 5a and the second surface 3 can be tapered or rounded, so as to avoid the presence of sharp edges in the articulation area with a further component present in the articular cavity in question.

The acetabular spacer device 1 further comprises an apex 6, placed at the top of the acetabular spacer device 1 and/or of the first surface 2. The apex 6 is opposite to the base 5.

The central symmetry axis X of the device 1 passes at the apex 6.

At the terminal circular perimeter of the first surface 2 there is an annular tab 8. The annular tab 8 extends externally with respect to the acetabular spacer device 1.

The tab 8 projects outwardly from the surface 2 by a portion Z.

In particular, the annular tab 8 and/or the section Z extends substantially perpendicular to the first surface 2 and/or coplanar with respect to the base 5 of the acetabular spacer device 1.

The annular tab 8 acts as an abutment or support for the patient's articular seat and/or acetabular bone. In this way, the stability of the positioning or implantation of the acetabular spacer device 1 is increased.

The acetabular spacer device 1 may have a hemisphere shape, and in this case the base 5 corresponds to the equatorial plane of the sphere which corresponds to the acetabular spacer device 1 or it may continue beyond it, and the base 5 will in this case be smaller than that of the equatorial plane of the sphere that corresponds to the acetabular spacer device 1. Alternatively, the acetabular spacer device 1 may have a smaller shape than that of a hemisphere, and the base 5 also in this case will have a smaller area than that of the equatorial plane of the sphere which corresponds to the acetabular spacer device 1.

Equatorial plane means a plane perpendicular to the central symmetry axis X of the articular component 2 and passing through a diameter thereof.

As already mentioned, the acetabular spacer device 1, or rather the first surface 2 thereof, comprises a plurality of elongated projections or recesses 4. These elongated projections or recesses extend towards the outside or towards the inside of the acetabular spacer device 1, with respect to the surface 2 itself.

The elongated projections or recesses 4 substantially depart from the apex 6 or from the area surrounding the apex 6, up to the terminal peripheral area of the first surface 2 and/or the annular tab 8.

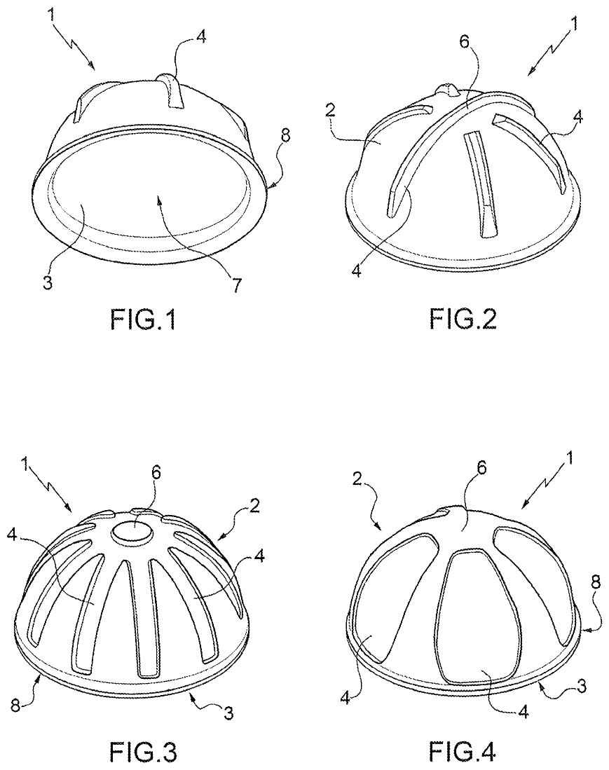

In one version, for example shown in FIGS. 1, 2 and 5-9, the first surface 2 has elongated projections 4, which extend towards the outside of the acetabular spacer device 1, therefore—in use—towards the bone of the patient.

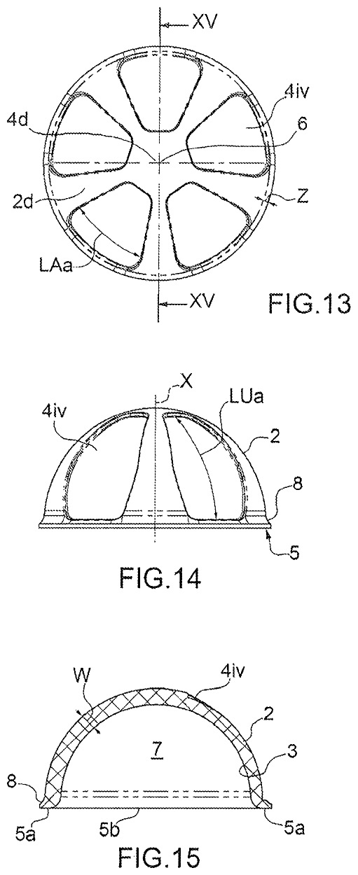

In the second and third versions of the acetabular spacer device 1, for example shown respectively in FIGS. 3, 10-12 and 4, 13-15, the first surface 2 has elongated recesses 4, which extend towards the inside of the device acetabular spacer 1, therefore—in use—away from the patient's bone.

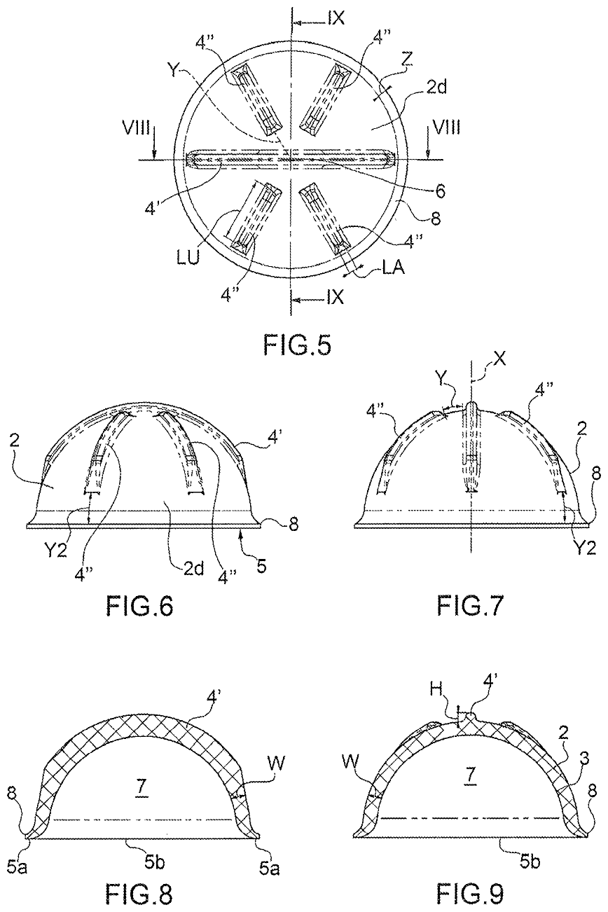

In the version for example shown in FIG. 5, it is seen that a first elongated projection 4′ extends along a maximum circumference section of the acetabular spacer device 1. The first projection 4′, therefore, has the shape of an axis of circumference that passes through the apex 6. The other or second elongated projections 4″ develop in a radial pattern from the apex 6.

In particular, in a version, these second elongated projections 4″ branch off from an area surrounding the apex 6, that is to say, they are spaced from it by a portion Y, as can be seen in FIG. 5. As can be seen in FIG. 7, the section Y is a curved section which follows the pattern of the first surface 2.

The first and second projections 4′, 4″ have an elongated shape, that is to say, they have a length LU greater than their width LA. They can have a substantially rectangular shape, possibly with rounded corners.

Moreover, the ends of the first and second projections 4′, 4″ are rounded or tapered towards the surface 2, and/or connected thereto in a degrading manner, in order to avoid the presence of sharp edges at the surface of the acetabular spacer device 1 which will come into contact with the bone tissue.

Moreover, the first and second projections 4′, 4″ extend upwards with respect to the first surface 2 by a section or height H.

In at least one embodiment example, H is substantially corresponding to the measure LA while the measure LU is much greater than the measure of LA or H.

For example, for the second projections 4″ LU is at least 5 times LA and the first projection 4′ has a measure equal to twice or thrice the second projection 4″. In one version of the invention (not shown), there may be two first projections 4′, placed perpendicularly with respect to one another or in an incident manner with respect to each other at the apex 6.

As can be seen in the accompanying figures, the projections 4′, 4″, at least in one embodiment, are equally spaced from one another.

The end of the first and second protrusions 4′, 4″ (opposite to that present at the apex 6) terminates at the terminal perimeter area of the first surface 2 and/or of the annular tab 8 and, in particular, at a distance Y2 with respect to the same.

As it can be seen, for example, in FIGS. 8 and 9, the thickness W of the acetabular spacer device 1 is given by the thickness of the wall constituting the device itself, and therefore by the distance between its first surface 2 and its second surface 3.

This shape of the elongated projections 4′, 4″ delimits corresponding angular sectors 2d of surface 2, having a substantially triangular shape, having a vertex at the area of the apex 6 and having as a base a section of the terminal perimeter of the first surface 2 itself. Each angular sector 2d is delimited laterally by two elongated projections 4″ or by a first projection 4′ and by a second projection 4″.

In one version of the invention, this thickness W is substantially constant along the entire extension of the acetabular spacer device 1.

As can be seen in FIG. 8, the projections 4′, 4″ are made in one piece with the acetabular spacer device 1 and therefore the latter, corresponding to the areas in which the projections 4′, 4″ are positioned, has a thickness equal to W+H.

The first projection 4′ substantially cuts the first surface 2 of the acetabular spacer device 1 into two same halves.

In the embodiment illustrated by way of example only, in the device 1 there is a projection 4′ and two projections 4″ for each half (of the acetabular spacer device 1) identified precisely by the projection 4′, for a total of four second projections 4″.

Alternatively, from 2 to 10 second projections 4″ may be present, possibly arranged symmetrically with respect to the first projection 4′.

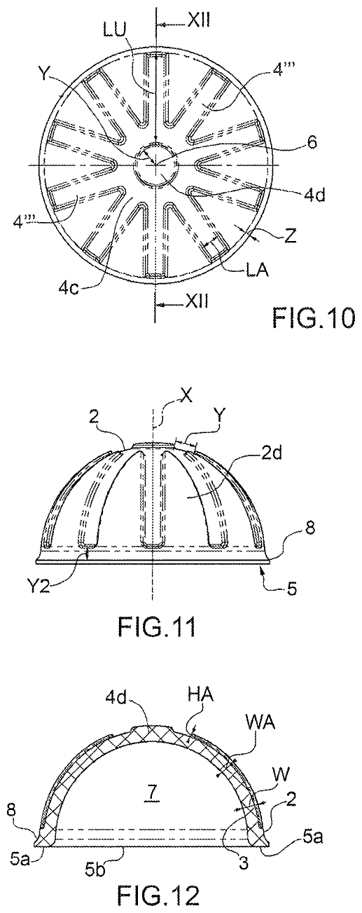

In the embodiment in FIGS. 4 and 10-12, as said, the first surface 2 has elongated recesses 4, which are retracted towards the inside of the acetabular spacer device 1. Considering the version in FIG. 10, the recesses 4 comprise elongated recesses 4′″ having a substantially rectangular shape, possibly with rounded corners.

As visible, for example, in FIG. 12, therefore, the thickness W of the acetabular spacer device 1 is equal to WA at the areas in which the recesses 4 are positioned, with WA smaller than W. In particular, the recesses extend towards the inside with respect to the first surface 2 by a section HA. Thus, the recesses 4 of the version in FIG. 3 have a depth HA. Therefore, WA is equal to the measure of W reduced by HA.

The end closest to the vertex 6 of each elongated recess 4′ branches off at a distance Y from the vertex 6.

The recesses 4′ are arranged radially around the apex 6 and, in at least one version of the invention, are equidistant from one another.

Therefore, as can be seen in the accompanying figures, the recesses 4′ have the shape of a recessed groove having a curved shape, substantially corresponding to that of the first surface 2 but recessed with respect to the latter.

The positioning of the recesses 4′ is such that their ends closest to the vertex 6 are close to each other, delimiting a sort of recessing groove 4c around the area of the apex 6, having a substantially circular and annular shape.

At the apex 6, in this way, a zone 4d of the first surface 2, having a substantially circular shape, is delimited.

This shape of the elongated recesses 4′ delimits corresponding angular sectors 2d of surface 2, having a substantially triangular shape, having a vertex at the groove 4c and having as a base a section of the terminal perimeter of the first surface 2 itself. Each angular sector 2d is delimited laterally by two elongated recesses 4′.

In the illustrated version, only by way of example, in the acetabular spacer device 1 there are ten elongated recesses 4′. Alternatively, there may be present from 6 to 16 elongated recesses 4′.

The recesses 4′, where not expressly indicated, substantially have the same features of the projections 4, 4′, 4″ of the previously described version, but instead of being projecting towards the bone, they are recessed with respect to the first surface 2.

Finally, in an analogous manner, in the embodiment shown in FIGS. 4 and 13-15, elongated recesses 4iv are shown, which have the same features as the previously described version but vary in shape.

In fact, the recesses 4iv have a substantially polygonal shape, for example trapezoidal or ovoid, in which the width LAa grows away from the apex 6 towards the circular terminal perimeter of the first surface 2 and/or towards the circular tab 8. In the other versions, the width LA of the projections or recesses 4′, 4″, 4′″ was substantially constant along their whole extension.

Furthermore, the elongated recesses 4iv have a length LUa substantially equal to 1-2 times the maximum width LAa (that considered at the circular terminal perimeter of the first surface 2) thereof.

As described above, at the apex 6 there is a zone 4d, of first surface 2, having a circular or polygonal shape, whose number of sides—in this last case—is equal to the number of elongated recesses 4iv.

In fact, in the area of the apex 6, the ends of the recesses 4iv comprise a side (for example the smaller base of a trapezium) of the polygon which constitutes the recess 4iv itself.

Moreover, the elongated recesses 4iv define angular sectors 2d, of first surface 2, having a substantially triangular or trapezoidal shape, with a vertex or smaller base at the area 4d and greater base at a portion of the circular terminal perimeter of the first surface 2. In order to perform its infection healing and treatment function, the acetabular spacer device 1 comprises at least one pharmaceutical or medical substance, such as for example an antibiotic.

The material constituting the acetabular spacer device 1, therefore, is a porous material and/or provided with canaliculi, capable of absorbing and/or eluting such at least one pharmaceutical or medical substance, and then releasing it in contact with the bone tissue in which an infection has developed.

Since that according to the present invention is a temporary acetabular spacer device 1, it must not remain permanently inside the human body. Therefore, it must be removed once it has performed its healing function against possible infections that arise at the implantation site. Therefore, any pores and/or canaliculi present therein have dimensions such as to prevent growth of the bone tissue within them. Therefore, in at least one version of the invention, any pores and/or canaliculi are smaller than 100 microns or micrometers.

One of the functions of the pores and/or canaliculi, for example, is that of allowing (by capillarity) the absorption and/or elution of the pharmaceutical or medical substances present or which can be added in the acetabular spacer device 1.

With regard to the latter aspect, in fact, it is known that a spacer, in order to eradicate the infection, has to release the pharmaceutical or medical substance adapted to the purpose in contact with the whole area of bone tissue to be treated, continuously for a certain period. Such (even small) amounts of substance, homogeneously released on the whole infected surface, act more efficiently, also with respect to larger amounts of antibiotic, whether distributed as lavage (and therefore having a short duration) and in a non-homogeneous way (and therefore not by placing an antibiotic device in contact with the whole infected zone).

The acetabular spacer device 1, as seen, being shaped similarly to the bone cavity in which it is to be implanted, and releasing the pharmaceutical or medical substance in a homogeneous continuous manner and of adequate extension, is suitable for performing this function effectively.

The acetabular spacer device 1 according to the invention is fixed to the bone tissue of the cavity in which it is to be inserted by bone cement, for example a bone cement comprising polymethyl methacrylate (PMMA) and/or similar polymers.

The projections or recesses 4 present on the first surface 2 aid the connection with the bone tissue as they determine the presence of spaces or areas in which the bone cement is somehow forced to remain, thus implementing the fixation of the device itself. In fact, the pressure exerted by the surgeon during the operation to fix and position the acetabular spacer device 1 in its special seat can cause the movement of the bone cement placed therein. This cement, in fact, can be squashed by the spacer device and move along its peripheral portions, leaving the apical area covered and/or in contact only with a light layer of bone cement.

Thanks to the projections or recesses 4, instead, large areas of the surface 2 of the spacer device remain in contact with a suitable layer of bone cement, which remains almost trapped between one projection 4′, 4″ and the other or inside of the recesses 4′, 4iv, thus facilitating the gluing or fixing of the device in the implantation site.

Furthermore, the projections or recesses 4 are positioned in or delimit some areas in which a greater resistance of the acetabular spacer device 1 is necessary, thus ensuring greater strength to the implantation area and avoiding the risk that the femoral or humeral head or the corresponding prosthetic or spacer components implanted at the respective cavities or joints can break through the (often thin) bone tissue found in those areas. The acetabular spacer device 1 according to the present invention is made of biologically compatible material, such as ceramic or plastic.

With regard to plastics, it may comprise at least one thermoplastic polymer, an acrylic resin, an acrylic polymer and/or copolymer, polymethyl methacrylate, a bone cement comprising polymethyl methacrylate and/or a similar polymer.

With regard to ceramics, the material may comprise a ceramic bone cement, optionally comprising calcium sulphate (CaSO.sub.4), or other components containing calcium.

In an alternative embodiment of the invention, the acetabular spacer device 1 can be made of a biologically compatible plastic and/or polymeric material such as for example polyethylene or high density polyethylene or ultra high molecular weight polyethylene (UHMWPE). In this case, the acetabular spacer device 1 can be made of such material or comprise an insert made of such material, for example placed at the second surface 3 thereof.

In fact polyethylene, and in particular UHMWPE, has self-lubricating properties and therefore increases the smoothness and/or implements the articulation with the complementary component mentioned above.

In particular, in fact, the cavity 7 is suitable for housing the head of the femur or the humerus or the head (substantially hemispherical or spherical) of the stem component (complementary to the acetabular spacer device 1) of a prosthesis or an implantable spacer device at the other end of the joint for the human body in question.

In a still alternative embodiment of the invention, the acetabular spacer device 1 is made of a composite material, that is to say, it can be made of one or more of the biologically compatible materials indicated above and comprises, inside it, a reinforcement or core (not illustrated) having a shape substantially corresponding to that of the acetabular spacer device 1, i.e. for example a cup shape.

Such internal reinforcement or core is made of a material which is structurally and/or mechanically more resistant than those constituting the outer portions of the acetabular spacer device 1, i.e., a different polymeric material or a metallic material.

In this version, the plurality of elongated projections 4 can be made in one piece with said biologically compatible material placed externally with respect to the reinforcement or core.

In a non-limiting version of the invention, the acetabular spacer device 1 can be entirely made of a plastic material or bone cement or polymethyl methacrylate.

Moreover, the biologically compatible material, as mentioned, is suitable to be additived and/or can be additived with one or more pharmaceutical or medical products, such as for example one or more antibiotics, for the reasons indicated above, for example gentamicin, vancomycin, etc. or other active ingredients. In fact, the acetabular spacer device 1, whatever the material with which it is made, comprises (and is able to elute in use, once implanted in the bone cavity in question) at least one pharmaceutical or medical substance, such as for example an antibiotic.

Such at least one pharmaceutical or medical substance may be present inside the acetabular spacer device 1 and/or be admixed thereto just before the implantation, depending on the patient's needs.

In fact, the porosity of the material allows it to be impregnated, for example by capillarity, and the subsequent release of these substances (for example prepared in the form of an aqueous solution).

One of the advantages of at least one version of the present invention is that the acetabular spacer device 1 is made of the same material as the bone cement used for fixing it to the bone tissue.

For example, if the bone cement comprises PMMA, also the biologically compatible material with which the spacer device is made comprises PMMA.

In this way, in addition to the advantages indicated above, the material constituting the acetabular spacer device 1 partially melts in contact with the bone tissue and in this way a very stable fixing between it and the bone tissue is determined, precisely by means of the cement bone that remains “trapped” in contact with its first surface 2.

It is therefore clear that the present invention guarantees an excellent and stable fixation with the bone tissue.

Furthermore, the spacer device 1 is made in one piece and therefore also the projections or recesses 4 are made in one piece with the device itself.

This also implements the stability of connection with the surrounding bone tissue.

From the foregoing description it is thus clear that the acetabular spacer device according to the present invention is able to replace any possible acetabulum of infected prostheses, preserving the articular space, an articulation surface having sufficient resistance to avoid break-through by (for example) the femoral head or corresponding components or bones in other joints of the human body and, being antibiotic-loaded and/or comprising pharmaceutical or medical substances, to treat the current infection. Furthermore, the acetabular spacer device, being fixed to the bone tissue with a bone cement that is in turn antibiotic-loaded, acts as a local release device of at least one pharmaceutical or medical substance and/or of an antibiotic (for example Gentamicin and/or Vancomycin, etc.), promoting effective therapy to eradicate the bone infection. In this way, it is also possible to strengthen the antibiotic action of the bone cement by delivering a larger amount with the acetabular spacer device or by delivering on site a substance different from that contained in the bone tissue, expanding the coverage and/or the action spectrum against any bacteria present at the site of infection, causing the various substances to act in a synergistic way.

The device, moreover, due to its hollow hemisphere geometry, allows the articulation with the spherical head of the complementary component of the bone tissue or of the prosthesis or of the stem component of a spacer, for example of the hip, possibly of a modular type.

Therefore, the perfect geometry allows a very stable articulation with the aforementioned components, such as with the head of a hip spacer, eliminating the risks of dislocation, this risk being frequent using only the stem component of a hip spacer. Furthermore, in this case, since the spherical head of the bone or prosthesis or of the spacer device, such as the stem component of the hip spacer, does not articulate directly on the bone of the articular cavity but on the second surface of the acetabular spacer device according to the present invention, the pain felt by the patient during the articulation is reduced and, as previously mentioned, the risk of breaking the acetabular roof—which is present in the absence of the acetabular spacer device itself—is eliminated.

Finally, the acetabular spacer device 1 according to the invention is preformed, i.e., it has a certain shape and conformation and is ready for use, without the need for the surgeon to resize, smooth or otherwise adapt it to anatomical dimensions or patient needs.

Finally, for this purpose, the acetabular spacer device 1 can be provided in several sizes, having different diameters or sections, as well as apical dimensions of a given height, so as to better adapt to the various anatomical shapes and sizes of the patient.

In particular, the acetabular spacer device 1 can be provided in 3-4 sizes. This is because it can mate with a prosthetic or spacer head (or their stem components) in an approximate way, at least in some variants thereof.

Again, at least in a version of the invention, the projections or recesses 4 are placed symmetrically with respect to a plane passing through the central symmetry axis X of the acetabular spacer device 1.

In a version of the invention, the projections or recesses 4 are positioned substantially radially with respect to the apex 6 of the acetabular spacer device 1.

In this case, the elongated projections or recesses 4 determine and/or delimit some channels, also arranged radially with respect to the apex 6 of the acetabular spacer device 1, which uniformly guide the distribution of the bone cement along the first surface 2, thereby implementing fixation thereof to the acetabular spacer device 1 and to the patient's bone.

The invention thus conceived is susceptible to numerous modifications and variations, all of which are within the scope of the inventive concept.

The features presented for a version or embodiment may be combined with the features of another version or embodiment, without departing from the scope of protection of the present invention.

Moreover, all of the details may be replaced by other technically equivalent elements. In practice, the materials used as well as the contingent size and shapes may be any according to the requirements, without departing from the scope of protection of the following claims.

The invention claimed is:

1. An acetabular spacer device adapted to be implanted in a bone cavity at a joint of a human body, the acetabular spacer device comprising:
a body made from polymethylmethacrylate and having,
a substantially hemispherical cup shape,
an outer first surface that is convex and is configured to be positioned in the bone cavity, and
an inner second surface that is concave and that defines an inner cavity; and
a pharmaceutical or medical substance adapted to treat an ongoing infection in the bone cavity, the pharmaceutical or medical substance being provided within the body of the acetabular spacer device,
wherein the inner second surface is configured to be articulated with a head of a stem component that is present at the joint of the human body, or with a head of a stem of a permanent prosthesis or of a spacer device configured to be arranged at the joint of the human body,
wherein the outer first surface comprises a plurality of elongated recesses or projections that have perimeters that each defines a linear segment or a trapezoidal shape or a substantially rectangular shape and that extend inwardly or outwardly in relation to the outer first surface;
a base, an apex of the outer first surface being opposite to the base at a top point of the outer first surface; and
an annular tab disposed at the base or at a terminal circular perimeter of the outer first surface, the annular tab extending outwardly for a distance in relation to the outer first surface and/or to the elongated recesses or projections,
wherein the annular tab provides an abutment or support for a patient's articular seat or acetabular bone, so as to increase stability of positioning or implantation of the acetabular spacer device.

2. The acetabular spacer device according to claim 1, wherein the annular tab extends radially further than the outer first surface and the plurality of elongated recesses or projections.

3. The acetabular spacer device according to claim 1, wherein the elongated recesses or projections depart from the apex or from a zone surrounding the apex and extend toward or to the circular terminal perimeter of the outer first surface, or have a radial arrangement in relation to the apex.

4. The acetabular spacer device according to claim 3, wherein the plurality of elongated recesses or projections comprise a first elongated projection and second elongated projections that extends outwardly, wherein the second elongated projections are radially disposed with respect to the apex and are spaced therefrom by a distance, and wherein the first elongated projection further extends along a portion of an outer circumference of the outer first surface crossing the apex or along an arc of circumference of the outer first surface crossing the apex.

5. The acetabular spacer device according to claim 1, wherein the plurality of elongated recesses or projections comprise elongated recesses that extend inwardly and that are radially arranged in relation to the apex and/or to a recessing groove defined around the apex.

6. The acetabular spacer device according to claim 5, wherein the recessing groove and the elongated recesses define angular sectors of the outer first surface having a triangular shape with a vertex at the recessing groove and a base at a portion of the circular terminal perimeter of the outer first surface.

7. The acetabular spacer device according to claim 1, wherein the plurality of elongated recesses or projections comprise elongated recesses having a polygonal or ovoidal shape.

8. An acetabular spacer device adapted to be implanted in a bone cavity at a joint of a human body, the acetabular spacer device comprising:
a body made from polymethylmethacrylate, the body having a substantially hemispherical cup shape and defining,
an outer first surface that is convex and configured to be positioned in the bone cavity,
an inner second surface that is concave and defines an inner cavity,
a base, and
an apex opposite to the base and positioned at a top point of the outer first surface,
wherein the outer first surface defines a plurality of elongated recesses or projections that extend inwardly or respectively outwardly in relation to the outer first surface,
wherein the plurality of elongated recesses or projections have a radial arrangement in relation to the apex and depart from the apex or from a surrounding zone of the apex, and
wherein the inner second surface is configured to be articulated with a head of a stem component that is present at the joint of the human body, or with a head of a stem of a corresponding permanent prosthesis or spacer device of the joint of the human body.

9. The acetabular spacer device according to claim 8, wherein the body comprises a pharmaceutical or medical substance arranged therein to be eluted in the bone cavity.

10. The acetabular spacer device according to claim 8, further comprising an annular tab extending outwardly of the base and/or of the elongated recesses or projections.

11. The acetabular spacer device according to claim 8, wherein the plurality of elongated recesses or projections extend longitudinally along the outer first surface for at least a portion of a distance between the apex and the base and have longitudinal axes that meet at the apex.

12. The acetabular spacer device according to claim 11, wherein the plurality of elongated recesses or projections extend longitudinally along the outer first surface for only a portion of a distance between the apex and the base and have longitudinal axes that meet at the apex.

13. The acetabular spacer device according to claim 12, wherein the plurality of elongated recesses or projections extend longitudinally along the outer first surface and are spaced both from the apex and the base.

14. The acetabular spacer device according to claim 11, wherein the plurality of elongated recessed or projections are configured as longitudinal ribs extending outwardly of the outer first surface.

15. The acetabular spacer device according to claim 11, wherein the elongated recesses or projections are configured as longitudinal cavities.

16. The acetabular spacer device according to claim 11, wherein the elongated recesses or projections define triangular portions of the outer first surface therebetween.

17. The acetabular spacer device according to claim 11, wherein the elongated recesses or projections define therebetween trapezoidal portions of the outer first surface, the trapezoidal portions having a smaller upper base and a larger lower base, the smaller upper base being closer to the apex than the larger lower base.

18. The acetabular spacer device according to claim 8, wherein the base of the body is perpendicular to a longitudinal axis of the body.

19. The acetabular spacer device according to claim 8, wherein the acetabular spacer device consists of said body.
